(19) United States
(12) Patent Application Publication
(10) Pub. No.: US 2026/0306700 A1
(43) Pub. Date: Oct. 8, 2026

(54) ANTIMICROBIAL BONE CEMENT COMPOSITIONS: METHODS OF MAKING AND USING THE SAME

(71) Applicant: Zonova Technologies Ltd, London (GB)

(72) Inventor: Georgia FLEET, London (GB)

(73) Assignee: Zonova Technologies Ltd, London (GB)

(21) Appl. No.: 19/641,325

(22) Filed: Apr. 7, 2026

Related U.S. Application Data
(60) Provisional application No. 63/784,710, filed on Apr. 7, 2025.

(51) Int. Cl. A61L 24/00; A61L 24/02

(52) U.S. Cl. CPC A61L 24/0015; A61L 24/02; A61L 2300/404; A61L 2430/02

(57) ABSTRACT

The disclosure provides antimicrobial bone cement compositions comprising a plurality of metallic nanoparticles, a photosensitizer, and a bone cement, which is mixed to form a metallic nanoparticle-photosensitizer bone cement mixture. This mixture exhibits activity in dark conditions without requiring a light source. The disclosure also provides methods of manufacturing an antimicrobial implant with the antimicrobial metallic nanoparticle-photosensitizer bone cement mixture compositions disclosed by the invention.

### CROSS REFERENCE TO RELATED APPLICATIONS

This application claims the benefit of and priority to U.S. Provisional Application No. 63/784,710, filed on Apr. 7, 2025, which is incorporated by reference in its entirety.

### FIELD

This invention relates generally to antimicrobial compositions and uses thereof, wherein the antimicrobial compositions are used in bone cement to prevent infection.

### BACKGROUND

Patients who receive biomaterials through orthopedic surgery have a high risk of developing deep infections. These biomaterials, including bone cement, are at risk of being contaminated with bacteria, which can then form a biofilm on the surface. Antimicrobial therapies that are currently in clinical use remain limited in their ability to effectively treat and prevent biofilm-related infections. Antibiotic-loaded bone cement has been previously used in clinical settings as a means to reduce bacterial infections in joint arthroplasties and joint replacements. However, as Bistolfi et. al. describes, multiple issues can still arise and lead to bacterial infection, including antibiotic resistance, modification of mechanical properties to allow for the antibiotic, release of the antibiotic, effectiveness, and toxicity (Bistolfi A, et al. ISRN Orthop. (2011)).

Photosensitizers are light-sensitive agents that absorb and transfer the energy of photons to surrounding molecules. This can lead to the formation of reactive oxygen species and free radicals, which are able to damage and kill bacteria. The mechanism of action does not lead to the selection of antibiotic-resistant bacteria, making it an attractive option for the prevention of bacterial infection. Examples commonly used are toluidine blue O and methylene blue. These compounds, when irradiated with certain wavelengths, can kill a wide range of microbes.

U.S. Pat. No. 8,580,309B2 describes antimicrobial mixtures comprising charge-stabilized metallic nanoparticles and a photosensitizer, and their use as light-activated antimicrobials. The authors described metallic nanoparticle-ligand-photosensitizer conjugates for use as light-activated antimicrobials capable of killing or preventing the growth of microbes on surfaces in a medical environment. This antimicrobial technology relies on nanoparticles to enhance the natural antimicrobial activity of the photosensitizer dye, which has not in the past exhibited its own antimicrobial activity. However, these photosensitizer dyes are activated by light and, therefore, are not active in the dark.

Bone cement, typically composed of polymethyl methacrylate (PMMA), acts as a structural grout that fills the space between a prosthetic implant and the living bone to ensure stable fixation. While it provides immediate mechanical stability, the surgical site is inherently vulnerable to Periprosthetic Joint Infection (PJI), a serious complication where bacteria form a resilient biofilm on the implant's surface. Integrating antibacterial agents into the cement is crucial because systemic antibiotics often struggle to reach these deep-seated areas effectively. Therefore, there is a need for antimicrobial bone cement that can be activated within a subject's body without requiring a light source for activation.

Presented herein are antimicrobial bone cement compositions that are active in both light and dark conditions, eliminating the need for a light source, and are suitable for use in bone cement for implanted medical devices.

### SUMMARY

In accordance with the purpose(s) of this invention, as embodied and broadly described herein, this invention, in some aspects, relates to an antimicrobial bone cement composition comprising a) a plurality of metallic nanoparticles; b) one or more photosensitizers; c) bone cement; wherein the metallic nanoparticles, one or more photosensitizers, and the radical initiator are incorporated into the bone cement to form an antimicrobial bone cement composition; wherein the antimicrobial bone cement composition exhibits antimicrobial activity under light and dark conditions.

In some embodiments, the antimicrobial bone cement composition of claim 1, wherein the plurality of metallic nanoparticles are selected from a group consisting of zinc oxide, zinc, gold, silver, copper, cupric oxide, magnesium oxide, iron (II,III) oxide, graphene oxide, titanium dioxide, tungsten (VI) oxide nanoparticles, alloys of two or more of these metals, or a combination thereof. In some embodiments, neither the plurality of metallic nanoparticles nor the one or more photosensitizers leaches from the composition. In some embodiments, the one or more photosensitizers comprises at least 0.001% by weight of the total polymer. In some embodiments, the metallic nanoparticles comprise about 0.1% to about 10% by weight of the total polymer. In some embodiments, the composition generates reactive oxygen species in the absence of an external light source.

In some embodiments, the one or more photosensitizers are selected from the group consisting of toluidine blue O, methylene blue, dimethylmethylene blue, zinc phthalocyanine, silicon phthalocyanine, aluminum phthalocyanine, rose bengal, porphyrin, haematoporphyrin, deuteroporphyrin, tin chlorin e6, poly-lysine derivatives of tin chlorin e6, m-tetrahydroxyphenyl chlorin, benzoporphyrin derivatives, tin etiopurpurin, bacteriochlorins, phenazines, acriflavine, proflavin, acridine orange, aminacrine, texaphyrins, cyanines including merocyanine 540, anthracyclins including adriamycin and epirubicin, pheophorbides, sapphyrins, fullerene, perylenequinonoid pigments including hypericin and hypocrellin, gilvocarcins, terthiophenes, benzophenanthridines, tris(2,2′-bipyridine)ruthenium(II) hexafluorophosphate, quercetin hydrate, curcumin, Zn(II) phthalocyanine, eosin-Y disodium salt, psoralens and riboflavin arianor steel blue, tryptan blue, crystal violet, azure blue cert, azure B chloride, azure 2, azure A chloride, azure B tetrafluoroborate, thionin, azure A eosinate, azure B eosinate, azure mix sicc, and azure II eosinate, or a combination thereof. In some embodiments, the photosensitizer is tris(2,2′-bipyridine)ruthenium(II) hexafluorophosphate, quercetin hydrate, curcumin, Zn(II) phthalocyanine, crystal voilet, azure-A, eosin-Y disodium salt, methylene blue, rose bengal, or a combination thereof. In some embodiments, the antimicrobial bone cement composition comprises zinc oxide and methylene blue, wherein the zinc oxide and methylene blue are dispersed within the bone cement.

In some embodiments, the plurality of nanoparticles comprises nanoparticles of less than 100 nm in diameter. In some embodiments, the composition further comprises a radiopaque filler to make the implantable device easily visible under X-rays

In another aspect, the present disclosure relates to a method for making an antimicrobial bone cement composition of claim 1 comprising the steps of: a) providing a cement powder comprising: i) a polymer powder; ii) a polymerization initiator; iii) a plurality of metallic nanoparticles; and iv) one or more photosensitizers; b) providing a liquid component comprising a polymerizable monomer and an activator; c) combining the cement powder with the liquid component to form a bone cement mixture; d) transferring the bonce cement mixture into a mold; and e) allowing the bone cement mixture to cure; wherein the cured bone cement mixture exhibits antimicrobial activity under both light and dark conditions.

In some embodiments, the initiator is a peroxide. In some embodiments, the cement powder further comprises a radiopaque filler to make the implantable device easily visible under X-rays. In some embodiments, transferring the antimicrobial bone cement mixture into a mold is completed within 10 minutes. In some embodiments, the mold is a bone cavity within a subject in need of a bone cement composition. In some embodiments, the antimicrobial bone cement is transferred into a mold via injection. In some embodiments, the antimicrobial bone cement is used in orthopedic or dental applications.

In another aspect, the present invention relates to a kit for producing the antimicrobial bone cement composition, comprising: polymethyl methacrylate, an initiator, a plurality of metallic nanoparticles, one or more photosensitizers, methyl methacrylate, and dimethyl-p-toluidine. In some embodiments, the kit further comprises one or more additives selected from zirconium oxide, barium sulfate, vitamin E, chemotherapy agents, or a combination thereof.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIG. 1 is a bone cement sample in the shape of a centrifuge tube used as a mold.

FIG. 2 shows extruded materials with varying photosensitizers. From left to right, FIG. 2 shows: bone cement (without any methylene blue, ZnO, or replacements), MB, Tris(2,2′-bipyridine)ruthenium(II) hexafluorophosphate, Quercetin hydrate, Curcumin, Zn(II) Phthalocyanine, Protoporphyrin, Crystal Voilet, Azure-A, and Eosin Y disodium salt.

FIG. 3 shows extruded materials with varying metallic nanoparticles. From left to right, FIG. 3 shows: bone cement (without any methylene blue, ZnO, or replacements), ZnO, silver nanoparticles, copper nanoparticles, cupric oxide nanoparticles, magnesium oxide nanoparticles, tungsten (VI) oxide nanoparticles, iron (II,III) oxide nanoparticles, graphene oxide, and titanium dioxide nanoparticles.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an,” and “the” include plural references unless the context clearly dictates otherwise. For example, reference to a component is intended also to include the composition of a plurality of components. References to a composition containing “a” constituent are intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

The term “subject,” “subjects,” “subjects in need thereof,” and “individuals” includes humans as well as non-human subjects. It will be understood that the subject to which a compound of the invention is administered need not suffer from a specific traumatic state. Indeed, the compounds of the invention may be administered prophylactically. The terms “subject,” “subjects,” “subjects in need thereof,” and “individuals” can be used interchangeably herein.

As used herein, the term “dark conditions” means away from direct light sources. Dark conditions are those in which the antimicrobial composition is not exposed to an external light source sufficient to activate a photosensitizer via photonic excitation. Dark conditions may include environments with minimal or incidental ambient light and are not limited to complete darkness. As used herein, the term “light source” refers to a source of light such as sunlight, visible light, laser light, or any other light of any specific wavelength. The term ‘dark conditions” may be used interchangeably with the term “in the dark.”

As used herein, the term “bone cement” refers to a polymeric material suitable for use in orthopedic or dental applications to secure, anchor, or stabilize an implant or fill a bone cavity. In some embodiments, bone cement comprises polymethyl methacrylate (PMMA) or copolymers thereof, along with polymerization initiators, activators, and optional additives.

As used herein, the term “pH agent” refers to a chemical used to change the pH of a solution. A base may be used to make a solution basic. The basic pH agent may be selected from NaOH, sodium bicarbonate, potassium hydrogen carbonate, potassium hydroxide, lithium hydroxide, calcium hydroxide, strontium hydroxide, barium hydroxide, ammonia, ethylamine, pyridine, aniline, ethylamine, or ammonium hydroxide. An acid may be used to make a solution acidic. The acidic pH agent may be selected from hydrochloric acid, phosphoric acid, nitric acid, perchloric acid, hydrobromic acid, sulfuric acid, hydroiodic acid, nitrous acid, formic acid, acetic acid, hydrocyanic acid, hydrogen sulfide, and hydrofluoric acid.

As used herein, the term “photosensitizer” refers to a substance capable of absorbing light and transferring energy to desired reactants. Photosensitizer refers to a compound capable of generating reactive chemical species, including reactive oxygen species and/or free radicals, either upon exposure to a light source or, in some embodiments of the present invention, in the absence of light when combined with one or more metallic nanoparticles.

As used herein, the term “sterilization” refers to a method for sterilizing medical devices and/or pharmaceuticals. Sterilization refers to a process for substantially reducing or eliminating viable microorganisms from a medical device or composition, including but not limited to bacterial, viral, and fungal species.

### II. Compositions

### A. Nanoparticle-Photosensitizer Conjugates

Provided herein are antimicrobial nanoparticle-photosensitizer bone cement compositions for medical devices. Also provided is a method of making an antimicrobial bone cement implants using a metallic nanoparticle-photosensitizer bone cement mixture. The method involves the incorporation of a metallic nanoparticle, such as zinc oxide, a photosensitizer dye, a radical initiator, and a polymethylmethacrylate bone cement into an implantable device. When incorporated into bone cement, the compositions impart antimicrobial activity under both light and dark conditions.

In some embodiments, the antimicrobial composition comprises a metallic nanoparticle-photosensitizer mixture that is dispersed within a polymeric bone cement matrix. In other embodiments, the metallic nanoparticles and the photosensitizer are present as separate components within the bone cement composition.

### 1. Metallic Nanoparticles

The term “nanoparticles” or “metallic nanoparticles” as used herein, refers to nanoparticles comprising a metal, metal oxide, alloy, or combinations thereof. In some embodiments, the metallic nanoparticles have at least one dimension in the range of about 1 to about 100 nm. In some embodiments, the nanoparticles used have a diameter of from about 1 to about 30 nm. In some embodiments, the nanoparticles have a diameter of from about 2 to about 5 nm. In other embodiments, the nanoparticles have a diameter of from about 10 to about 25 nm. In yet another embodiment, the nanoparticles have a diameter from about 15 to about 20 nm.

Nanoparticles typically, but not exclusively, comprise metals. They may also comprise alloys of two or more metals, or more complex structures such as core-shell particles, rods, stars, spheres, or sheets. A core-shell particle may typically comprise a core of one substance, such as a metal, metal oxide, or silica, surrounded by a shell of another substance, such as a metal, metal oxide, or metal selenide. The term “metallic” as used herein is intended to encompass all such structures having a metallic outer surface.

In some embodiments, the outer surface of the metallic nanoparticles of the present invention comprises a main group metal or transition metal, such as cobalt. In other embodiments, the metallic nanoparticles comprise one or more of zinc, gold, silver, and/or copper nanoparticles, and/or alloys of two or more of these metals. Metallic nanoparticles suitable for use in the present compositions may include, but are not limited to, nanoparticles comprising zinc, zinc oxide, silver, copper, cupric oxide, magnesium oxide, iron (II,III) oxide, tungsten (VI) oxide, titanium dioxide, graphene oxide, gold, alloys thereof, or combinations thereof. In some embodiments, the nanoparticles are zinc nanoparticles. In preferred embodiments, the metallic nanoparticles are zinc oxide particles or alloys thereof.

The metallic nanoparticles of the present invention may be chosen such that, when attached via the ligand to the photosensitizer to form the metallic nanoparticle-photosensitizer mixture, the mixture generates singlet oxygen and/or free radicals. In some embodiments, the mixture generates both singlet oxygen and free radicals. In particular embodiments, zinc oxide nanoparticles are selected due to their ability to generate reactive oxygen species and their favorable compatibility with polymeric bone cement compositions.

Singlet oxygen generation may be measured by assay: several such methods are known to those skilled in the art, for example, photoluminescence. Free radical generation may be measured using electron proton resonance (EPR).

Examples of metallic nanoparticles that may be suitable are nanoparticles having a diameter greater than about 2 nm, which exhibit plasmon resonance in the wavelength band of about 200 to about 1600 nm, i.e., covering the visible to near infrared bands. The plasmon resonance may be measured by UV spectroscopy. It may be seen for both the free and conjugated nanoparticle. In some embodiments, nanoparticles will exhibit plasmon resonance at wavelengths of from about 500 to about 600 nm.

Another property that may be used to help select a suitable nanoparticle is the molar extinction coefficient of the conjugated photosensitizer. When a photosensitizer is conjugated via a ligand to a suitable nanoparticle, the extinction coefficient of the photosensitizer may be enhanced, compared to the extinction coefficient that would be expected based on an equivalent concentration of the photosensitizer alone. Without wishing to be bound by theory, it is thought that this enhancement occurs because the photosensitizer coordinates to the surface of the nanoparticle. Thus, in order to select suitable nanoparticles, the extinction coefficient of the conjugate could be measured using a spectrophotometer. Any enhancement is acceptable. In some embodiments, the extinction coefficient may range anywhere from about 2 to about 30 times or more; in others, it may range from about 5 to about 30 times or more; in others, it may range from about 10 to about 30 times or more, and in others, it may range from about 20 to about 30 times or more, compared to what is expected based on the same concentration of the unconjugated photosensitizer.

In some embodiments, the outer surface of the nanoparticles may comprise one or more of the group comprising zinc, gold, silver, or copper. In some embodiments, the nanoparticles may comprise one or more of the group comprising zinc or alloys thereof. Suitable alloys may also comprise one or more other metals such as silver, copper, gold, aluminum, or combinations thereof.

In other embodiments, the nanoparticles described in the preceding paragraph comprise core-shell particles. It is possible for such core-shell particles to comprise a magnetic core or magnetic layer. An example of such a magnetic core-shell particle is a particle having a magnetic core and an outer shell that comprises gold. In some embodiments, the nanoparticles are zinc nanoparticles.

### 2. Antimicrobial Photosensitizers

A photosensitizer is a compound that can be excited by light of a specific wavelength. Thus, such a compound may have an absorption band in the ultraviolet, visible, or infrared portion of the electromagnetic spectrum and, when the compound absorbs radiation within that band, it generates cytotoxic species, thereby exerting an antimicrobial effect. The effect may be due to the generation of singlet oxygen, but the invention is not limited to photosensitizers that exhibit antimicrobial effects through the generation of singlet oxygen.

It is a requirement of the present invention that the photosensitizer is chosen such that, when attached to the metallic nanoparticle-ligand core to form the conjugate, the conjugate generates singlet oxygen and/or free radicals. Preferably, the conjugated photosensitizer generates both singlet oxygen and free radicals without the presence of a light source. Singlet oxygen and free radical generation may be measured as described above.

Without wishing to be bound by theory, it is thought that the photosensitizer and nanoparticles are associated via dative covalent bonds, wherein the electrons are provided by, for example, S or N moieties on the photosensitizer.

In some embodiments, the photosensitizer demonstrates antimicrobial activity within a subject's body, without a need to be activated by a light source. In some embodiments, the photosensitizer is one or more photosensitizers selected from the group comprising: phenothiaziniums (e.g. toluidine blue O, methylene blue, dimethylmethylene blue), phthalocyanines (e.g. zinc, silicon and aluminum phthalocyanines), halogenated xanthenes (e.g. rose bengal), porphyrins (e.g. haematoporphyrin derivatives, deuteroporphyrin), chlorins (e.g. tin chlorin e6, poly-lysine derivatives of tin chlorin e6, m-tetrahydroxyphenyl chlorin, benzoporphyrin derivatives, tin etiopurpurin), bacteriochlorins, phenazines (e.g. neutral red), acridines (e.g. acriflavine, proflavin, acridine orange, aminacrine), texaphyrins, cyanines (e.g. merocyanine 540), anthracyclins (e.g. adriamycin and epirubicin), pheophorbides, sapphyrins, fullerene, perylenequinonoid pigments (e.g. hypericin, hypocrellin), gilvocarcins, terthiophenes, benzophenanthridines, psoralens and riboflavin arianor steel blue, tryptan blue, crystal violet, azure blue cert, azure B chloride, azure 2, azure A chloride, azure B tetrafluoroborate, thionin, azure A eosinate, azure B eosinate, azure mix sicc, and azure II eosinate. In one embodiment, particularly preferred photosensitizers are methylene blue, or rose Bengal. In some embodiments, methylene blue is selected as a photosensitizer due to its demonstrated antimicrobial activity under both light and dark conditions when combined with suitable metallic nanoparticles.

a. Organic Photosensitizers

The photosensitizers may be organic (including dyes, porphyrins, chlorins, furocoularins, xanthenes or monoterpene) or non-organic (including gaseous mercury, quantum dots, or nanowires/nanorods). In the disclosed invention, the photosensitizers are organic as they are favorable for absorption through the swelling process.

Amongst the list of organic photosensitizers, three main families with similar mechanisms can be identified.

i. Porphyrins and Chlorins.

In some embodiments, the photosensitizer is categorized as a porphyrin or chlorin photosensitizer. These photosensitizers include porphyrin, porfimer, temoprfin, and talaporfin sodium. Porphyrin and porphyrin-like molecules constitute the most common type of photosensitizer; they are all built around the porphyrin centre:

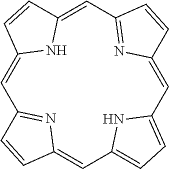 [SMILES: C1=Cc2cc3ccc(cc4nc(cc5ccc(cc1n2)[nH]5)C=C4)[nH]3]

This molecule, thanks to its highly conjugated structure, can absorb photonic energy to enter an excited state commonly referred to as Sn (where n is the level of excitation, n=0 being the ground state). Each level of energy is stored in the higher molecular orbitals (moving from HOMO to LUMO) of the molecule and can be transferred to the orbital of triplet oxygen to excite it to a singlet state. Singlet oxygen is highly reactive to organic species and promotes the formation of peroxo bridges, which in turn promote the formation of radicals, also known as ROS (Reactive oxygen species).

The absorption of photons by the porphyrin is determined by the energy bandgap between the HOMO and LUMO orbitals. Various strategies can be developed to affect that bandgap, mostly by modifying the π-conjugated network and adding or replacing some heteroatoms. The resulting effect can determine the energy of the photon that is necessary to induce an excited state of porphyrin.

One strategy that can be employed is the addition of a metal-based core to the center of the porphyrin. This is commonly referred to as metalloporphyrin and is largely inspired by the structure of hemes in hemoglobin. These molecules are generally synthesized ex situ but could be formed in situ under the right conditions.

It has been observed that under certain conditions, porphyrin could reach an excited state, not through the direct absorption of a photon but through the transfer of excitement via another molecule (enzymes, protein) (Afonso S G, Enríquez de Salamanca R, Batlle A M. Braz J Med Biol Res. 1999 March; 32(3):255-66).

Therefore, it is possible that a cooperative effect between the porphyrin (or porphyrin-like compound) and a metal nanoparticle could, under the right circumstances, catalyse the formation of ROS without the need for photon absorption.

ii. Dyes and Xanthenes

In some embodiments, the photosensitizer is categorized as a dye and xanthenes photosensitizer. These photosensitizers include acridine orange, methylene blue, toluidine blue O, Rose Bengal, and erythrosine. This family presents a structure that conjugates π bonds around a benzenic center. In its oxidized state, the dye's benzenic ring (one or more) is not saturated and instead displays a 1,4-cyclohexadiene structure linked to double bonds in positions 3,6. Those double bonds can be linked directly or via conjugation to a heteroatom presenting non-binding doublets (e.g., O, N, S). The driving mechanism for the reduction is the completion of the benzene ring and its stabilization via aromaticity. This is initiated by the loss of two (or more) electrons and the bonding of one or more of the terminal heteroatoms to a H atom from the environment around the dye. This transformation ends with the more thermodynamically stable reduced form of the dye.

This process is entirely reversible should the reduced dye be exposed to a strong oxidizer (more thermodynamically reactive than the oxidized dye) and the dye being in a medium allowing proton exchange (e.g. water). This applies directly to the examples disclosed herein with methylene blue. Along with the aromatization of a benzene ring, the heteroatom can be involved in the closing (during the reduction) of a heterocycle. This applies directly to the examples disclosed herein, including those with Rose Bengal (Hai, Yue & Feng, Shan & Wang, Lili & Ma, Yetao & Zhai, Yiran & Wu, Zijun & Zhang, Sichao & He, Xin. (2017). Coordination Mechanism and Bio-Evidence: Reactive Y-Ketoenal Intermediated Hepatotoxicity of Psoralen and Isopsoralen Based on Computer Approach and Bioassay. Molecules. 22. 1451. 10.3390/molecules22091451).

Because of this highly conjugated structure, the band gap between HOMO and LUMO is such that the molecule can absorb a photon and enter an excited state. This energy can then be transferred to molecular oxygen to transform it from triplet oxygen to singlet oxygen, leading to the creation of ROS

However, it should also be mentioned that, because of their natural oxidative properties (and their reversible nature), this family of molecules can demonstrate antibacterial activity in the absence of a photon.

iii. Exotic Photosensitizers

In some embodiments, the photosensitizer is categorized as an exotic photosensitizer. These photosensitizers include azulene and psoralen. This family is less defined and regroups mostly seldomly used photosensitizers. These molecules tend to usually bind to certain sites under the effect of photon absorption and therefore are non-catalytic and don't present a specific mechanism. Based on their undefined properties, this category is not included in the disclosed compositions and methods. The photosensitizer categories that are used in the disclosed compositions and methods are: Porphyrins, Chlorins, Dyes, and Xanthenes. Other photosensitizers can be listed and added individually based on their structure and mechanism of action. The proportion of metallic nanoparticles to photosensitizers may vary.

The metallic nanoparticle-photosensitizer mixture may also comprise further components. In some embodiments, it may have a targeting moiety associated with it. The targeting moiety can be associated with the conjugate via any suitable means; for example, it may be attached to the nanoparticle core, to the ligand, or to the photosensitizer. Such targeting moieties may be suitable, for example, for targeting specific microorganisms or for targeting cancer cells. For example, they may be antibodies with specificity for the target organism or cancer cell. Other examples of targeting moieties include bacteriophages, protein A (targets Staphylococcus aureus), and bacterial cell-wall binding proteins or peptides.

The novel nanoparticle: photosensitizer compositions have been found to demonstrate particularly effective antimicrobial properties. Thus, all uses of mixtures as described herein apply to the novel compositions described herein.

### B. Metallic Oxides and Photosensitizer Mixtures

Provided herein are antimicrobial compositions comprising metallic oxides and photosensitizer mixtures. In some embodiments, the antimicrobial composition comprises a metallic oxide and a photosensitizer mixed directly into the bone cement without requiring pre-formed conjugates. In certain embodiments, the metallic oxide is zinc oxide and the photosensitizer is methylene blue (MB). These metallic oxide-photosensitizer mixtures are capable of generating antimicrobial activity in both light and dark conditions and are particularly well-suited for incorporation into polymethyl methacrylate bone cement compositions. Zinc oxide (ZnO) is an inorganic compound that typically can be found in the form of a powder. ZnO can be directly added to the nanoparticle-photosensitizer polymer mixture for extrusion or silicone molding/injection. Zinc oxide is known to generate reactive oxygen species (Guo, et al., Toxicol In Vitro, 27(2):731-738 (2013)). Ions do not leach out when ZnO is not in water. If submerged in water and at an acidic pH, it may begin to leach Zn+ ions.

Complexes of methylene blue with the metal form of precious metals (most likely nanoparticles) can be favoured due to the availability of the S doublet (divalent sulphur). That includes affinity for Ag, or Au and potentially other thiophilic compounds.

In some embodiments, it is possible that the beneficial effect of the addition of both a metallic nanoparticle and a photosensitizer relies on the different mechanisms of action, the photosensitisers through ROS (oxygen radicals) formation leading to cell/DNA oxidation, the metal nanoparticles through membrane accumulation/permeation leading to apoptosis or through reactive oxygen species generation (Bruna T, Maldonado-Bravo F, Jara P, Caro N. Int J Mol Sci. 2021 Jul. 4; 22(13):7202). Silver nanoparticles exhibit ion release, or leaching, which provide their method for killing microorganisms.

Additional metallic oxides suitable for use in such mixtures include, but are not limited to, magnesium oxide, cupric oxide, iron oxide, tungsten oxide, titanium dioxide, and combinations thereof. Similarly, copper, cupric oxide, and magnesium oxide can be directly added to the nanoparticle-photosensitizer polymer mixture for extrusion or silicone molding/injection but exhibit ion release. Iron oxide nanoparticles can release ions at acidic pH but not at a pH of about 7.

The following nanoparticles can be directly added to the nanoparticle-photosensitizer polymer mixture for extrusion or silicone molding/injection and exhibit reactive oxygen species release: Tungsten (VI) (Popov, et al., Mater. Sci. Eng. C. Mater. Biol. Appl. 108:110494 (2020)); graphene oxide; titanium dioxide.

### 1. Mechanisms of Reactivity of Methylene Blue in the Dark

In the presence of light, photons with a specific energy level can activate MB. Once activated, MB can react with “classic” oxygen, also called triplet oxygen (³O₂), to form singlet oxygen (¹O₂). The difference between these two types of oxygen lies in the outer electron shell configuration.

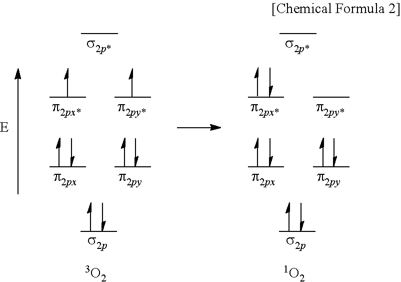

Having an empty orbital results in increased reactivity of singlet oxygen to facilitate oxidation steps, such as the oxidation of lipids or proteins that constitute the bacterial cell wall and lead to their destruction.

In this case, MB acts as a catalyst and is regenerated under its oxidated form (blue).

In the example below, we can see the oxidation by singlet oxygen of a C—C bond in dioxethane leading to its separation in 2 units along with the catalytic cycle of MB (Lee, J. Bioluminescence, the Nature of the Light, (2020) University of Georgia).

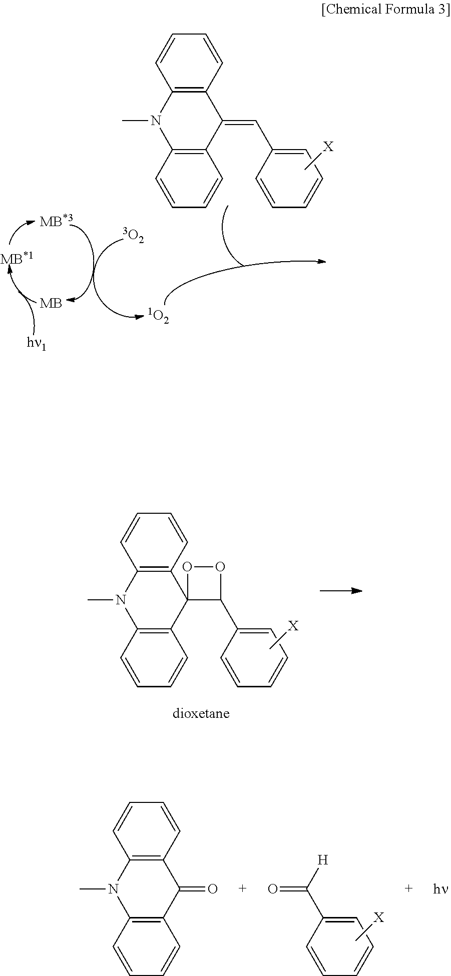

In the dark, the absence of photons means there is no basis for the catalytic cycle of the MB and therefore no formation of singlet oxygen. However, in basic or acidic conditions, the reaction of the MB in its oxidized form (blue) with organic species (such as bacterial cell wall) is catalyzed, the source of electrons and protons being the bacteria themselves. This reaction, however, is not reversible and will progressively lead to the reduction of the MB in leucomethylene blue (LMB) (reduced form, colorless) (Mahlum, J D et al., J. Phys. Chem. C 2021, 125, 17, 9038-9049).

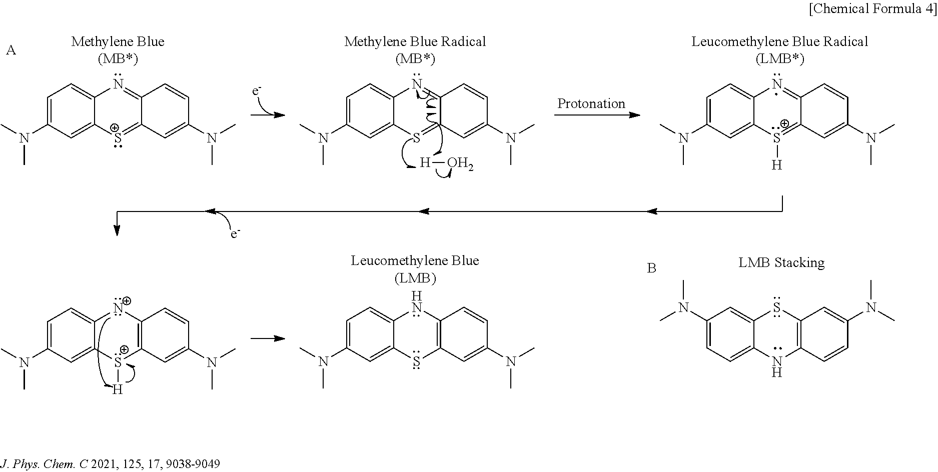

In some embodiments, we can expect the presence of zincate (in the form of an MB-zincate complex) to act as the catalyst as a basic source (equilibrium zincate⇔zinc oxide+hydroxide ion).

It is expected, according to this theory, that the MB would progressively become inactive as it is reduced, but this is not observed visually (the reduction from blue to colorless) in practice when samples are exposed to bacteria in light conditions. Depending on the concentration encapsulated, the time to inactivity could vary (based on flow rate and exposure to reducing agents, such as bacteria). As such high concentrations are encapsulated, it is expected this is why this reduction in activity has not been observed in experiments. However, there is a possibility that dissolved oxygen could lead to the oxidation of LMB even without light activation. This can be observed in the famous blue bottle experiment (The ‘blue bottle’ experiment in association with the Nuffield Foundation).

### C. Bone Cement

The antimicrobial compositions described herein are incorporated into bone cement compositions suitable for orthopedic or dental use. In some embodiments, bone cement can comprise a polymer powder and the monomer capable of undergoing radical polymerization. In some embodiments bone cement mixture is produced by mixing the polymer powder component and the liquid monomer component. The antimicrobial bone cement compositions described herein comprise at least a polymer powder, a plurality of metallic nanoparticles, one or more photosensitizers, a radical initiator, an activator, and a monomer, which may be mixed in various concentrations to yield antimicrobial bone cement compositions. In some embodiments, the monomer is methyl methacrylate. In some embodiments, the bone cement may comprise a radiopaquer or radiopaque filler, an activator, a polymerization initiator, and/or a polymerization accelerator, and optionally, additional additives. The present invention relates to antimicrobial bone cement comprising the bone cement described above, with the addition of a plurality of metallic nanoparticles and one or more photosensitizers.

In some embodiments, polymethylmethacrylate (PMMA) is used as the polymer powder. In other embodiments, suitable polymers include copolymers of methyl methacrylate and one or more monomers that can be copolymerized with it, including methylacrylate, styrene, and ethylacrylate.

In some embodiments, the metallic nanoparticles and the photosensitizer are used in about equal percentages by weight. In some embodiments, the metallic nanoparticles and the photosensitizer are used in different percentages by weight. In some embodiments, the combination of the metallic nanoparticles and the photosensitizer comprises about 0.001% to about 10% of the total polymer weight. In some embodiments, the bone cement composition comprises at least 0.001% photosensitizer. In some embodiments, the photosensitizer composes about 0.001% to about 20%, 0.001% to 15%, 0.001% to 10%, 0.001% to 5%, 0.001% to 2%, 0.05% to about 20%, 0.05% to 15%, 0.05% to 10%, 0.05% to 5%, 0.05% to 2%, 0.1% to 20%, 0.1% to 15%, 0.1% to 10%, 0.1% to 5%, 0.1% to 2%, 0.5% to 5%, 0.5% to 2%, 1% to 5%, 1% to 2% of the total polymer weight. In some embodiments, the nanoparticles comprise about 0.05% to about 20%, 0.05% to 15%, 0.05% to 10%, 0.05% to 5%, 0.05% to 2%, 0.1% to 20%, 0.1% to 15%, 0.1% to 10%, 0.1% to 5%, 0.1% to 2%, 0.5% to 5%, 0.5% to 2%, 1% to 5%, 1% to 2% of the total polymer weight. In some embodiments, the combination comprises about 2% of the total polymer weight. In some embodiments, the combination comprises about 4% of the total polymer weight. In some embodiments, the composition comprises 1% metallic nanoparticles and 1% photosensitizer. In some embodiments, the composition comprises 2% metallic nanoparticles and 2% photosensitizer.

In some embodiments, the antimicrobial bone cement comprises an activator that is selected from the group consisting of N,N-dimethyl-p-toluidine, N,N-bis-hydroxyethyl-p-toluidine, trioctylmethylammoniumchloride, N,N-dimethyl-aniline, tetrabutylammonium chloride, lithium chloride, saccharin, 1,8-diazabicyclo[5.4. Oundec-7-ene, and 1,5-diazabicyclo(4.3.0)non-5-ene, phthalimide, maleimide, Succinimide, pyromellitic acid diamide, and a mixture of at least two thereof. In some embodiments, the activator is an aromatic amine.

In some embodiments, the activator is used in an amount of 0.1-5% by weight of the total bone cement mixture. In some embodiments, the activator is used in an amount of about 0.5% by weight of the total bone cement mixture.

In some embodiments, the bone cement further comprises a radical stabilizer. In some embodiments, the radical stabilizer is selected from p-hydroquinone, o-hydroquinone, 2,6-di-t-butyl-p-hydroquinone, 2-t-butyl-p-hydrochinone, and 2,6-di-t-butyl-4-methyl-phenol.

In some embodiments, the radical initiator is a polymerization initiator. In some embodiments, the initiator is a peroxide. The peroxide preferably comprises no free acidic groups. The peroxide can be an organic peroxide or an inorganic peroxide, such as, for example, dialkylperoxides or hydroperoxides. For example, the peroxide can be selected from the group consisting of dibenzoylperoxide, cumene-hydroperoxide, 1,1,3,3-tetramethylbutylperoxide, t-butylperoxide, t-amyl-hydroperoxide, di-isopropylbenzen-mono-hydroperoxide, and a mixture of at least two thereof. In some embodiments, the peroxide is benzoyl peroxide.

In some embodiments, the initiator is used in an amount of between 0.1 to 3% by weight relative to the bone cement.

In some embodiments, any number of additional additives are optionally added to the cement mixture. In some embodiments, additives may include radio-opacifying additives such as ceramic particles including barium sulfate, zirconium oxide, magnesium oxide, and barium sulfate may also be added. In some embodiments, antibiotic additives may be added, such as erythromycin, gentamicin, sodium oxacillin, sodium cefazolin powder, gentamicin powder, gentamicin sulphate, or vancomycin may be used. Vitamin E may be used as an additive. Additional additives in bone cement are known in the art (see Arora, et al., “Polymethylmethacrylate bone cements and additives: A review of the literature” World J Orthop. 4(2):67-74 (2013)).

In some embodiments, the bone cement mixture can contain further ingredients aside from the components mentioned above.

In some embodiments, a radiopaque filler, or radiopaquer, used interchangeably herein, may be added to make the device easily visible under X-rays.

The radiopaquer can be a common radiopaquer in this field. Suitable radiopaquers can be soluble or insoluble in the monomer for radical polymerization. The radiopaquer is preferably selected from the group consisting of metal oxides (such as, for example, Zirconium dioxide), barium Sulfate, toxicologically acceptable heavy metal particles (such as, for example, tantalum), ferrite, magnetite (Supra magnetic magnetite also, if applicable), and biocompatible calcium salts. Said radiopaquers preferably have a mean particle diameter in the range of 10 nm to 500 μm. Moreover, conceivable radiopaquers also include esters of 3.5-bis(acetamido)-2,4,6-triiodobenzoic acid, gadolinium compounds, such as gadolinium chelate involving the esters of 1,4,7,10 tetraazacyclododecan-1,4,7,10-tetraacetic acid (DOTA). In preferred embodiments, the radiopaque filler is zirconium dioxide.

### II. Methods of Making

The present disclosure provides methods for manufacturing antimicrobial bone cement compositions comprising metallic nanoparticles and one or more photosensitizers. The methods described herein yield bone cement compositions that exhibit antimicrobial activity under both light and dark conditions.

### A. Process for Preparation of the Antimicrobial Bone Cement

In some embodiments, the antimicrobial bone cement is produced first by mixing solids to produce a cement mixture. Such a mixture may be produced by combining the individual components; polymer powder, a plurality of metallic nanoparticles, one or more photosensitizers, and a radical initiator, the particular ingredients of which are described above. In some embodiments, the mix is shaken or ground to thoroughly homogenize. The solids are mixed at any suitable temperature below the freezing point. In some embodiments, the solutions are contacted at or about room temperature.

The cement mixture is mixed with a liquid mixture to form the bone cement. The liquid mixture comprises an activator and methyl methacrylate. In some embodiments, the liquid mixture comprises about 20-40% by weight of the total bone cement mixture.

In some embodiments, the liquid mixture comprises the plurality of nanoparticles and/or the one or more photosensitizers. In some embodiments, said components are solely added to the liquid mixture. In some embodiments, said components are added to both the solid and liquid mixture. In some embodiments, said components are added to the final bone cement mixture prior to hardening/curing.

In some embodiments, the liquids are mixed thoroughly and combined with the dry cement powder mixture to form a homogenized solution. In some embodiments, the solution containing the metallic nanoparticle-photosensitizer-radical initiator-bone cement mixture is transferred to a mold appropriate for forming a medical implant. In preferred embodiments, transferring the antimicrobial bone cement mixture into the mold is done within about 10 minutes. In some embodiments, the mold is then tapped or vibrated to remove air bubbles. In some embodiments, the antimicrobial bone cement mixture is allowed to stand at room temperature for about 10 minutes, about 10 minutes to about 1 hour, or about 1 hour to about 2 hours.

The pH of solutions may be such that no adjustment is required upon mixing, or the pH of the mixture may be controlled by the use of a suitable buffer.

The individual components may be mixed in any proportion, such that the desired concentration is achieved in the mixed solution. In one embodiment, the initial concentrations of each component are selected as required so that the desired concentration in the mixed solution is achieved when equal volumes of solid components and liquid components are mixed together.

The desired concentration of the nanoparticles in the mixture depends on the desired final concentration at the site to be treated. These concentrations have been discussed throughout the disclosure. This may vary, and a suitable choice depends both on the size of the nanoparticle and the concentration of the photosensitizer solution. In some embodiments, the final concentration of the nanoparticles in the mixture is from about 1×10¹¹ to about 5×10¹⁵ particles/ml. In some embodiments, the final concentration is from about 3×10¹¹ to about 1×10¹⁵ particles/ml. In order to obtain such a final concentration, the initial concentration of the nanoparticle solution is from about 1×10¹² to about 1×10¹⁶ particles/ml. If the nanoparticle solution, as prepared or as obtained commercially, is of a higher concentration than this, it may be necessary to dilute the nanoparticle solution before mixing with the photosensitizer. For example, an original nanoparticle solution containing 1×10¹⁴ or 1×10¹⁵ particles/ml may be diluted 1:10 to 1:100, such that the concentration before mixing with the photosensitizer solution is from 1×10¹² to 1×10¹⁴.

In various embodiments, the initial concentration of photosensitizer solution is chosen such that when mixed with the nanoparticle solution, the final concentration of photosensitizer at the treatment site is from about 500 to about 600 mg/L, 600 to about 700 mg/L, 700 to about 800 mg/L, 800 to about 900 mg/L, more preferably from about 800 mg/L.

It should be noted that the final concentration at the treatment site may not necessarily correspond to the concentration in the antimicrobial bone cement. For instance, in the treatment of periodontal pockets and wounds, the treatment site may be flooded with body fluid such as saliva or blood.

### III. Methods of Using

### A. Antimicrobial Effect of the Bone Cement

The bone cement mixtures of the present invention have an antimicrobial effect, i.e., they are capable of killing or inhibiting the growth of microorganisms, including bacteria, viruses, fungi, and prions, that can cause disease in humans, animals, or plants. In some embodiments, the mixtures of the present invention are used to kill or inhibit the growth of Staphylococcus aureus. Staphylococcus aureus as used in this application shall also include Methicillin-Resistant Staphylococcus aureus (“MRSA”). The mixtures of the present invention may also be used to kill or inhibit the growth of Propionibacterium acnes, Escherichia coli, and Candida albicans.

In other embodiments, the mixtures of the present invention are used to kill or prevent the growth of the microbes involved in wound infections and in disinfecting or sterilizing wounds and other lesions on a subject. Thus, the mixtures of the present invention may be used to kill or inhibit the growth of Streptococcus sanguis, Porphyromonas gingivalis, Fusobacterium nulceatum, Actinobacillus actinomycetemcomitans, Candida albicans, Streptococcus mutans and lactobacilli, Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis Bacteroides gingivalis, Bacteroides melaninogenicus (also known as Prevotella melaninogenica), Bartonella henselae, Bartonella quintana, Bordetella bronchiseptica Bordetella pertussis, Borrelia burgdorferi, Brucella abortus, Brucella melitensis, Brucella suis, Burkholderia, Burkholderia mallei, Burkholderia pseudomallei, Burkholderia cepacian, Calymmatobacterium granulomatis, Campylobacter coli, Campylobacter fetus, Campylobacter jejuni, Helicobacter pylori, Chlamydia trachomatis, Chlamydophila, Chlamydophila pneumoniae (also called Chlamydia pneumoniae), Chlamydophila psittaci (also called Chlamydia psittaci), Clostridium botulinum, Clostridium difficile, Clostridium perfringens (also called Clostridium welchii), Clostridium tetani, Corynebacterium, Corynebacterium diphtheriae, Corynebacterium fusiforme, Coxiella burnetiid, Ehrlichia chaffeensis, Ehrlichia ewingii, Eikenella corrodens, Enterobacter 17 5 10 15 20 25 30 35 cloacae, Enterococcus avium, Enterococcus durans, Enterococcus faecalis, Enterococcus faecium, Enterococcus gallinarum, Enterococcus maloratus, Escherichia coli, Fusobacterium necrophoru, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Leptospira interrogans, Leptospira noguchii, Listeria monocytogenes, Methanobacterium extroquens, Microbacterium multiforme, Micrococcus luteus, Moraxella catarrhalis Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma Mexican, Neisseria gonorrhoeae, Neisseria meningitidis, Pasteurella multocida, Pasteurella tularensis, Peptostreptococcus, Porphyromonas gingivalis, Prevotella melaninogenica (previously called Bacteroides melaninogenicus), Pseudomonas aeruginosa, Rhizobium radiobacter, Rickettsia prowazekii, Rickettsia psittaci, Rickettsia quintana, Rickettsia rickettsia, Rickettsia trachomae, Rochalimaea, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia Streptococcus agalactiae, Streptococcus avium, Streptococcus bovis, Streptococcus cricetus, Enterococcus faecium, Enterococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Ureaplasma urealyticum, Vibrio cholerae, Vibrio comma, Vibrio parahaemolyticus, Vibrio vulnificus, Wolbachia, Yersinia enterocolitica, Yersinia pestis, Yersinia pseudotuberculosis The bacteria may be selected from Acinetobacter baumannii, Pseudomonas aeruginosa, Enterobacteriaceae, Enterococcus faecium, Staphylococcus aureus, Helicobacter pylori, Campylobacter spp, Salmonellae, Neisseria gonorrhoeae, Haemophilus influenzae, Shigella spp. The bacteria may be a bacteria resistant to one or more antibiotics, for example a bacterium selected from Acinetobacter baumannii (carbapenem-resistant), 18 Pseudomonas aeruginosa (carbapenem-resistant), Enterobacteriaceae (carbapenem resistant, ESBL-producing), 5 Enterococcus faecium (vancomycin-resistant), Staphylococcus aureus (methicillin-resistant and/or vancomycin-intermediate and resistant), Helicobacter pylori (clarithromycin-resistant), Campylobacter spp., (fluoroquinolone-resistant), Salmonellae (fluoroquinolone resistant), Neisseria gonorrhoeae (cephalosporin-resistant and/or fluoroquinolone resistant), Streptococcus pneumoniae (penicillin-non-susceptible), Haemophilus influenzae (ampicillin-resistant), Shigella spp., (fluoroquinolone-resistant).

### B. Applications of Mixtures

The present disclosure provides the use of the compositions of the present invention in the manufacture of bone cement. Bone cement may be used to reduce microbial activity in tissues of a body cavity such as a bone wound.

In some embodiments, the antimicrobial bone cement compositions described herein are intended for use in a clinical environment. In some embodiments, the compositions may be finally shaped at the time of a surgical operation. In some embodiments, the compositions may be left to harden/cure within the body of a subject. The antimicrobial compositions described herein, in some embodiments of the present disclosure, exhibit antimicrobial activity instantly. As described herein, the compositions are shown to reach at least 2-log reduction >99% in antimicrobial activity against a microbe. In some embodiments, the antimicrobial bone cement is left in the body cavity indefinitely. In some embodiments, the antimicrobial bone cement compositions described herein do not substantially leach out any portion of the metallic nanoparticles or the photosensitizers.

The antimicrobial bone cement compositions are disclosed herein to be useful in orthopedic and dental applications. Applications of the antimicrobial bone cement compositions include joint replacements, spinal surgeries, fracture fixation, and bone tumor treatment, among others. In some embodiments, other therapeutic agents are added to the bone cement. In some embodiments, chemotherapeutic agents such as doxorubicin and cisplatin.

### EXAMPLES

### Example 1: Bacterial Testing of Samples

### Materials and Methods

### Day 0:

A single bacterial pellet was placed into a 15 mL sterile tube. A milliliter of Dulbecco's phosphate buffered saline (DPBS) was added to the pellet, and the pellet was allowed to fully dissolve over the course of 5 minutes. The solution was the vortexed for 10 seconds. A 100 μL aliquot was removed from the solution and placed on an agar plate. A spreader was use to spread an even layer of the solution around the plate. An agar plate box was sterilized by washing with 70% isopropyl alcohol (IPA), and a wetted blue roll was placed at the bottom of the box. The agar plate was placed into the box on a raised plastic platform and incubated.

### Day 1:

After incubation, individual colonies were collected, streak plated and incubated.

### Day 2:

A 10 mL aliquot of Nutrient broth was added to a centrifuge tube. A single colony from the plate prepared on Day 1 was collected and mixed into the nutrient broth, and the tube was vortexed for 10 seconds. The prepared tube was placed in an incubator at 250 rpm at a temperature of 37° C. overnight.

### Day 3:

The centrifuge tube from Day 2 was centrifuged at 3800 rpm for 10 minutes. The supernatant was removed, and 10 mL of DPBS was added. This mixture was vortexed for 10 seconds. This was repeated once.

Optionally, the bacterium was resuspended into 1 mL DPBS. A 100 μL aliquot was added to a container containing 1.9 mL DPBS. This was vortexed to mix. The absorbance of this solution was measured at 600 nm, and Table 1 was used to calculate an amount of DPBS needed to dilute the stock bacterium. 200 μL of stocks were measured into new containers, and the calculated volume of DPBS was added to each.

TABLE 1

| | | Volume of |
|---|---|---|
| | | DPBS to |
| Absorbance | Stock | add to |
| 600 nm | CFU/mL | stock (mL) |
| 0.667 | 7.79E+09 | 3.117 |
| 0.675 | 7.90E+09 | 3.160 |
| 0.661 | 7.71E+09 | 3.084 |

In instances where the absorbance was not measured, the stock was not diluted. In all instances, a 100 μL aliquot of the inoculum was added to a first well of a well plate. The inoculum was serially diluted ten times into wells each containing 120 μL PBS by taking 30 μL aliquots from the previous well. In duplicate, 5 μL of each was plated in a line on nutrient agar.

The desired sample was plated on a glass slide, and 25 μL of inoculum was added to the sample. This was placed in a humidity chamber overnight.

The sample bacterium tested against was selected from Escherichia coli (ATCC 8739), Candida albicans (ATCC 10231), and Staphylococcus epidermidis (ATCC 12228), as indicated in the Examples. A 25 μL inoculum was added to each sample, and spot plates were prepared with 5 μL at each dilution level.

### Example 2: Preparation of Antimicrobial Bone Cement

### Materials and Methods

The following components were added to a sealable container: polymethyl methylacrylate (PMMA) (10 g), benzyl peroxide (BPO) (89.3 mg), zinc oxide (ZnO) (125 mg), methylene blue (MB) (250 mg), and zirconium dioxide (ZrO₂) (2 g). These materials were mixed thoroughly until a fine, smooth, homogeneous powder was formed. To this was added methyl methacrylate (MMA) (5 g) and dimethyl-p-toluidine (DmpT) (80 mg). This was mixed until homogenous. This mixture was then transferred to a suitable mold and left to cure. Molds may be chosen based on testing requirements or application needs. The transfer to the mold should be performed within 10 minutes of mixing, as curing will begin. Bubbles were removed from the cement mixture by tapping the mold or by using a cement vibrator. The cement was cured for one hour at room temperature.

### Results

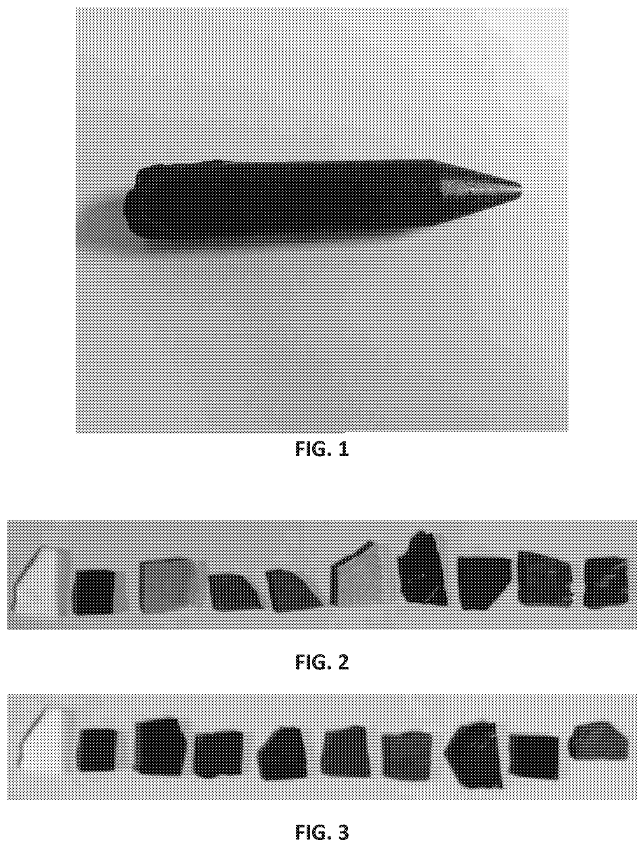

FIG. 1 shows an example bone cement sample after curing in the shape of a centrifuge tube used as a mold.

Various compositions of bone cement were tested for antibacterial properties, as outlined in Examples 3-5. In these Examples, the best-performing compositions were determined. For Gram positive bacteria (Staphylococcus epidermidis), the best composition was found to be methylene blue with zinc oxide. For Gram negative bacteria (Escherichia coli), the best compositions were found to be methylene blue with zinc oxide and crystal violet with zinc oxide. For fungi (Candida albicans), the best compositions were found to contain crystal violet and magnesium oxide.

### Example 3: Variation of Photosensitizer

Using the process as described in Example 2, the photosensitizer (methylene blue) was exchanged with the following compounds at the same weight percentage: 1) Tris(2,2′-bipyridine)ruthenium(II) hexafluorophosphate; 2) Quercetin hydrate; 3) Curcumin; 4) Zn(II) Phthalocyanine; 5) Protoporphyrin IX; 6) Crystal Voilet; 7) Azure-A; 8) Eosin-Y disodium salt. The products of this process are represented in FIG. 2, showing bone cement with MB replacements. From left to right, FIG. 2 shows: Bone cement (without any methylene blue, ZnO, or replacements), MB, Tris(2,2′-bipyridine)ruthenium(II) hexafluorophosphate, Quercetin hydrate, Curcumin, Zn(II) Phthalocyanine, Protoporphyrin, Crystal Voilet, Azure-A, and Eosin Y disodium salt.

Antibacterial testing was conducted with all materials described above, as described in Example 1. Antibacterial testing was carried out against Escherichia coli (ATCC 8739), Candida albicans (ATCC 10231), and Staphylococcus epidermidis (ATCC 12228). Each test was carried out in biological duplicate.

Samples tested for performance in the light were left on a table top within a room lit by LED lights at Ca. 250-350 Lux. Samples tested in the dark were left in the absence of light by placing them into a closed cabinet.

All test samples were left at room temperature overnight. Once complete, the solution was resuspended and serial dilutions were spot plated to determine bacterial numbers.

### Results

Each of Escherichia coli, Candida albicans, and Staphylococcus epidermidis were tested against the compositions described above in both light and dark conditions. Tables 2 and 3 describe testing against E. coli. Tables 4 and 5 describe testing against C. albicans. Tables 6 and 7 describe testing against S. epidermidis. In these results, the inoculum was a bacteria count without exposure to any sample. The control was a bacteria count after exposure to a control sample (i.e. a plain polymer with no methylene blue, zinc oxide, or replacements thereof).

TABLE 2
Testing against E. coli in light conditions.

| | Mean | | Standard |
|---|---|---|---|
| | % Log | Mean | deviation |
| Description | reduction | CFU/mL | CFU/mL |
| Inoculum | n/a | 4.22E+08 | 4.38E+07 |
| Control | n/a | 4.63E+07 | 3.03E+07 |
| Tris(2,2'-bipyridine) | >99.99% | 2.00E+02 | 0 |
| ruthenium(II) | (limit of | | |
| hexaflurophosphate | detection) | | |
| Quercetin hydrate | 90.61% | 3.90E+06 | 5.80E+05 |
| Curcumin | 97.00% | 1.40E+06 | 9.31E+05 |
| Zinc phthalocyanine | 97.69% | 9.82E+05 | 3.57E+05 |
| Protoporphyrin IX | >99.99% | 2.00E+02 | 0 |
| | (limit of | | |
| | detection) | | |
| Crystal violet | >99.99% | 2.00E+02 | 0 |
| | (limit of | | |
| | detection) | | |
| Azure A chloride | 99.95% | 2.12E+04 | 3.31E+03 |
| Eosin Y | >99.99% | 2.00E+02 | 0 |
| disodium salt | (limit of | | |
| | detection) | | |
| Methylene Blue | >99.99% | 2.00E+02 | 0 |
| | (limit of | | |
| | detection) | | |

TABLE 3
Testing against E. coli in dark conditions.

| | Mean | | Standard |
|---|---|---|---|
| | % Log | Mean | deviation |
| Description | reduction | CFU/mL | CFU/mL |
| Inoculum | n/a | 4.22E+08 | 4.38E+07 |
| Control | n/a | 1.03E+07 | 8.56E+05 |
| Tris(2,2'-bipyridine) | 82.90% | 2.04E+06 | 1.45E+06 |
| ruthenium(II) | | | |
| hexaflurophosphate | | | |
| Quercetin hydrate | 84.76% | 1.89E+06 | 1.49E+06 |
| Curcumin | 91.33% | 1.15E+06 | 1.03E+06 |
| Zinc phthalocyanine | 68.23% | 3.28E+06 | 3.96E+05 |
| Protoporphyrin IX | 99.43% | 3.47E+05 | 4.83E+05 |
| Crystal violet | >99.99% | 2.00E+02 | 0 |
| | (limit of | | |
| | detection) | | |
| Azure A chloride | 99.95% | 1.15E+04 | 1.48E+04 |
| Eosin Y | 99.79% | 2.20E+04 | 2.12E+03 |
| disodium salt | | | |
| Methylene Blue | >99.99% | 2.00E+02 | 0 |
| | (limit of | | |
| | detection) | | |

TABLE 4
Testing against C. albicans in light conditions.

| | Mean | | Standard |
|---|---|---|---|
| | % Log | Mean | deviation |
| Description | reduction | CFU/mL | CFU/mL |
| Inoculum | n/a | 1.76E+10 | 8.00E+8 |
| Control | n/a | 4.94E+07 | 4.28E+07 |
| Tris(2,2'-bipyridine) | 62.46% | 1.47E+07 | 2.08E+06 |
| ruthenium(II) | | | |
| hexaflurophosphate | | | |
| Quercetin hydrate | 0.00% | 7.21E+07 | 1.25E+07 |
| Curcumin | 0.00% | 7.80E+07 | 1.45E+07 |
| Zinc phthalocyanine | 38.84% | 2.96E+07 | 2.47E+07 |
| Protoporphyrin IX | 98.12% | 7.99E+05 | 4.49E+05 |
| Crystal violet | >99.99% | <2.00E+02 | 0 |
| | (limit of | | |
| | detection) | | |
| Azure A chloride | 99.88% | 4.94E+04 | 1.33E+04 |
| Eosin Y | 81.89% | 8.81E+06 | 7.47E+06 |
| disodium salt | | | |
| Methylene Blue | 94.24% | 2.30E+06 | 6.72E+05 |

TABLE 5
Testing against C. albicans in dark conditions.

| | Mean | | Standard |
|---|---|---|---|
| | % Log | Mean | deviation |
| Description | reduction | CFU/mL | CFU/mL |
| Inoculum | n/a | 3.83E+09 | 8.00E+8 |
| Control | n/a | 1.07E+08 | 3.31E+07 |
| Tris(2,2'-bipyridine) | 5.27% | 9.92E+07 | 5.31E+06 |
| ruthenium(II) | | | |
| hexaflurophosphate | | | |
| Quercetin hydrate | 24.23% | 7.94E+07 | 4.16E+06 |
| Curcumin | 55.64% | 6.93E+07 | 7.26E+07 |
| Zinc phthalocyanine | 36.54% | 6.70E+07 | 1.15E+07 |
| Protoporphyrin IX | 38.98% | 6.84E+07 | 3.43E+07 |
| Crystal violet | >99.99% | <2.00E+02 | 0 |
| | (limit of | | |
| | detection) | | |
| Azure A chloride | 75.43% | 3.17E+07 | 2.61E+07 |
| Eosin Y t | 40.91% | 6.60E+07 | 3.31E+07 |
| disodium sal | | | |
| Methylene Blue | 67.04% | 3.53E+07 | 1.03E+07 |

TABLE 6
Testing against S. epidermidis in light conditions.

| | Mean | | Standard |
|---|---|---|---|
| | % Log | Mean | deviation |
| Description | reduction | CFU/mL | CFU/mL |
| Inoculum | n/a | 7.81E+06 | 4.08E+06 |
| Control | n/a | 2.00E+04 | 1.70E+03 |
| Crystal Violet | 99% | <2.00E+02 | 0 |
| | (Limit of | | |
| | detection) | | |
| Eosin-Y | 99% | <2.00E+02 | 0 |
| | (Limit of | | |
| | detection) | | |
| Methylene Blue | 99% | <2.00E+02 | 0 |
| | (Limit of | | |
| | detection) | | |

TABLE 7
Testing against S. epidermidis in dark conditions.

| | Mean | | Standard |
|---|---|---|---|
| | % Log | Mean | deviation |
| Description | reduction | CFU/mL | CFU/mL |
| Inoculum | n/a | 7.81E+06 | 4.08E+06 |
| Control | n/a | 2.21E+06 | 1.20E+05 |
| Crystal Violet | 99.87% | 2.13E+04 | 2.98E+04 |
| Eosin-Y | 87.00% | 3.73E+05 | 3.37E+05 |
| Methylene Blue | >99.99% | | |
| | (Limit of | <2.00E+02 | 0 |
| | detection) | | |

### Example 4: Variation of Metallic Nanoparticle

Using the process as described in Example 2, the metallic nanoparticle (zinc oxide) was exchanged at the same percentage weight: 1) silver nanoparticles (<100 nm); 2) copper nanoparticles (40-60 nm); 3) cupric oxide nanoparticles (<50 nm); 4) magnesium oxide nanoparticles (50 nm); 5) tungsten (VI) oxide nanoparticles (<100 nm); 6) iron (II,III) oxide nanoparticles (50-100 nm); 7) graphene oxide powder (average number of layers 15-20); and 8) titanium dioxide nanoparticles (<100 nm). The products of this process are represented in FIG. 3, showing bone cement with ZnO replacements. From left to right, FIG. 3 shows: Bone cement (without any methylene blue, ZnO, or replacements), ZnO, silver nanoparticles, copper nanoparticles, cupric oxide nanoparticles, magnesium oxide nanoparticles, tungsten (VI) oxide nanoparticles, iron (II,III) oxide nanoparticles, graphene oxide, and titanium dioxide nanoparticles.

Antibacterial testing was conducted with all materials described above, as described in Example 1. Antibacterial testing was carried out against Escherichia coli (ATCC 8739), Candida albicans (ATCC 10231), and Staphylococcus epidermidis (ATCC 12228). Each test was carried out in biological duplicate.

Samples tested for performance in the light were left on a table top within a room lit by LED lights at Ca. 250-350 Lux. Samples tested in the dark were left in the absence of light by placing them into a closed cabinet.

All test samples were left at room temperature overnight. Once complete, the solution was resuspended and serial dilutions were spot plated to determine bacterial numbers.

Each of Escherichia coli, Candida albicans, and Staphylococcus epidermidis were tested against the compositions described above in both light and dark conditions. Tables 8 and 9 describe testing against E. coli. Tables 10 and 11 describe testing against C. albicans. Tables 12 and 13 describe testing against S. epidermidis.

### Results

TABLE 8
Testing against E. coli in light conditions.

| | | Mean | | Standard |
|---|---|---|---|---|
| | | % Log | Mean | deviation |
| Description | | reduction | CFU/mL | CFU/mL |
| Inoculum | | n/a | 4.85E+08 | 1.06E+07 |
| Control | | n/a | 1.75E+08 | 1.38E+08 |
| Silver | | >99.99% | <2.00E+02 | 0 |
| (<100 | nm) | (limit of | | |
| | | detection) | | |
| Copper | | >99.99% | <2.00E+02 | 0 |
| (40-60 | nm) | (limit of | | |
| | | detection) | | |
| Copper (II) Oxide | | >99.99% | <2.00E+02 | 0 |
| (<50 | nm) | (limit of | | |
| | | detection) | | |
| Magnesium Oxide | | >99.99% | <2.00E+02 | 0 |
| (<50 | nm) | (limit of | | |
| | | detection) | | |
| Tungsten (VI) Oxide | | >99.99% | <2.00E+02 | 0 |
| (<100 | nm) | (limit of | | |
| | | detection) | | |
| Iron (II, III) Oxide | | >99.99% | <2.00E+02 | 0 |
| (50-100 | nm) | (limit of | | |
| | | detection) | | |
| Graphene Oxide | | >99.99% | <2.00E+02 | 0 |
| | | (limit of | | |
| | | detection) | | |
| Titanium Dixide | | >99.99% | <2.00E+02 | 0 |
| | | (limit of | | |
| | | detection) | | |
| Zinc Oxide | | >99.99% | <2.00E+02 | 0 |
| (20 | nm) | (limit of | | |

detection)

TABLE 9
Testing against E. coli in dark conditions.

| | | Mean | | Standard |
|---|---|---|---|---|
| | | % Log | Mean | deviation |
| Description | | reduction | CFU/mL | CFU/mL |
| Inoculum | | n/a | 4.85E+08 | 1.06E+07 |
| Control | | n/a | 6.06E+08 | 1.40E+08 |
| Silver | | >99.99% | <2.00E+02 | 0 |
| (<100 | nm) | (limit of | | |
| | | detection) | | |
| Copper | | >99.99% | 5.88E+03 | 1.66E+03 |
| (40-60 | nm) | | | |
| Copper (II) Oxide | | 99.97% | 3.24E+05 | 3.74E+05 |
| (<50 | nm) | | | |
| Magnesium Oxide | | >99.99% | 2.00E+04 | 2.16E+04 |
| (<50 | nm) | | | |
| Tungsten (VI) Oxide | | 99.15% | 6.42E+06 | 5.48E+06 |
| (<100 | nm) | | | |
| Iron (II, III) Oxide | | 97.62% | 1.43E+07 | 6.16E+05 |
| (50-100 | nm) | | | |
| Graphene Oxide | | >99.99% | <2.00E+02 | 0 |
| | | (limit of | | |
| | | detection) | | |
| Titanium Dioxide | | 99.66% | 6.49E+06 | 8.72E+06 |
| Zinc Oxide | | >99.99% | <2.00E+02 | 0 |
| (20 | nm) | (limit of | | |

detection)

TABLE 10
Testing against C. albicans in light conditions.

| | | Mean | | Standard |
|---|---|---|---|---|
| | | % Log | Mean | deviation |
| Description | | reduction | CFU/mL | CFU/mL |
| Inoculum | | n/a | 1.76E+10 | 8.00E+08 |
| Control | | n/a | 2.49E+08 | 2.19E+08 |
| Silver | | 99.98% | 1.20E+05 | 1.63E+05 |
| (<100 | nm) | | | |
| Copper | | 99.99% | 7.05E+03 | 3.31E+03 |
| (40-60 | nm) | | | |
| Copper (II) Oxide | | 99.94% | 1.21E+05 | 3.83E+04 |
| (<50 | nm) | | | |
| Magnesium Oxide | | 99.43% | 1.13E+06 | 2.16E+05 |
| (<50 | nm) | | | |
| Tungsten (VI) Oxide | | 97.82% | 4.30E+06 | 7.47E+05 |
| (<100 | nm) | | | |
| Iron (II, III) Oxide | | 98.77% | 2.42E+06 | 1.74E+05 |
| (50-100 | nm) | | | |
| Titanium Dioxide | | 98.53% | 3.13E+06 | 1.74E+06 |
| Zinc Oxide | | 99.48% | 1.19E+06 | 8.88E+05 |
| (20 | nm) | | | |

TABLE 11
Testing against C. albicans in dark conditions.

| | | Mean | | Standard |
|---|---|---|---|---|
| | | % Log | Mean | deviation |
| Description | | reduction | CFU/mL | CFU/mL |
| Inoculum | | n/a | 3.83E+09 | 8.00E+07 |
| Control | | n/a | 7.24E+06 | 2.66E+06 |
| Silver | | 76.56% | 1.80E+06 | 1.05E+06 |
| (<100 | nm) | | | |
| Copper | | 78.42% | 1.76E+06 | 1.27E+06 |
| (40-60 | nm) | | | |
| Copper (II) Oxide | | 48.54% | 3.62E+06 | 5.31E+05 |
| (<50 | nm) | | | |
| Magnesium Oxide | | 92.52% | 5.24E+05 | 4.16E+04 |
| (<50 | nm) | | | |
| Tungsten (VI) Oxide | | 80.73% | 1.63E+06 | 1.29E+06 |
| (<100 | nm) | | | |
| Iron (II, III) Oxide | | 71.13% | 2.26E+06 | 1.45E+06 |
| (50-100 | nm) | | | |
| Titanium Dioxide | | 62.55% | 2.66E+06 | 5.80E+05 |
| Zinc Oxide | | 0.00% | 8.72E+06 | 7.24E+06 |
| (20 | nm) | | | |

TABLE 12
Testing against S. epidermidis in light conditions.

| | | Mean | | Standard |
|---|---|---|---|---|
| | | % Log | Mean | deviation |
| Description | | reduction | CFU/mL | CFU/mL |
| Inoculum | | n/a | 7.81E+06 | 4.08E+06 |
| Control | | n/a | 2.00E+04 | 1.70E+03 |
| Silver | | 99% | <2.00E+02 | 0 |
| (<100 | nm) | (Limit of | | |
| | | detection) | | |
| Copper (II) Oxide | | 99% | <2.00E+02 | 0 |
| (<50 | nm) | (Limit of | | |

detection)

TABLE 13
Testing against S. epidermidis in dark conditions.

| | | Mean | | Standard |
|---|---|---|---|---|
| | | % Log | Mean | deviation |
| Description | | reduction | CFU/mL | CFU/mL |
| Inoculum | | n/a | 7.81E+06 | 4.08E+06 |
| Control | | n/a | 2.21E+06 | 1.20E+05 |
| Silver | | >99.99% | | 0 |
| (<100 | nm) | (Limit of | | |
| | | detection) | <2.00E+02 | |
| Copper (II) Oxide | | >99.99% | <2.00E+02 | 0 |
| (<50 | nm) | (Limit of | | |

detection)

### Example 5: Antibacterial Testing: Lower Levels of Methylene Blue

Samples were prepared as described in Example 2, with PMMA (10 g), BPO (89.3 mg), ZnO (125 mg; 1% w/w), ZrO₂ (2 g), and MB (varying amounts: 62.5 mg, 125 mg, and 250 mg). Antibacterial testing was conducted as described in Example 1. The results of these assays are provided in Tables 14 and 15.

### Results

The results from testing lower levels of methylene blue in bone cement against E. coli indicate the concentration may be reduced at least to 0.5% while maintaining >99.99% reduction in E. coli in both light and dark conditions.

TABLE 14
Testing against E. coli in light conditions.

| | | Mean | | Standard |
|---|---|---|---|---|
| | | % Log | Mean | deviation |
| Description | | reduction | CFU/mL | CFU/mL |
| Inoculum | | n/a | 8.98E+08 | 3.95E+07 |
| Control | | n/a | 3.60E+08 | 2.12E+07 |
| 250 | mg MB | >99.99% | 1.28E+03 | 1.52E+03 |
| (2%) | | | | |
| 125 | mg MB | >99.99% | 1.28E+03 | 1.52E+03 |
| (1%) | | | | |
| 62.5 | mg MB | >99.99% | 1.06E+04 | 0 |
| (0.5%) | | | | |
| 30 | mg MB | >99.99% | 2.00E+02 | 0 |
| (0.24%) | | | (limit of | |
| | | | detection) | |
| 15 | mg BM | >99.99% | 2.00E+02 | 0 |
| (0.12%) | | | (limit of | |
| | | | detection) | |
| 7.5 | mg MB | >99.99% | 2.00E+02 | 0 |
| (0.06%) | | | (limit of | |
| | | | detection) | |
| 1 | mg MB | >99.99% | 2.00E+02 | 0 |

| | |
|---|---|
| (0.008%) | (limit of |
| | detection) |

TABLE 15
Testing against E. coli in dark conditions.

| | | Mean | | Standard |
|---|---|---|---|---|
| | | % Log | Mean | deviation |
| Description | | reduction | CFU/mL | CFU/mL |
| Inoculum | | n/a | 8.98E+08 | 3.95E+07 |
| Control | | n/a | 4.74E+08 | 5.73E+07 |
| 250 | mg MB | >99.99% | 2.00E+02 | 0 |
| (2%) | | | (limit of | |
| | | | detection) | |
| 125 | mg MB | >99.99% | 2.00E+02 | 0 |
| (1%) | | | (limit of | |
| | | | detection) | |
| 62.5 | mg MB | >99.99% | 2.00E+02 | 0 |
| (0.5%) | | | (limit of | |
| | | | detection) | |
| 30 | mg MB | 99.98% | 9.21E+05 | 1.27E+06 |
| (0.24%) | | | | |
| 15 | mg BM | 99.99% | 2.24E+04 | 5.02E+03 |
| (0.12%) | | | | |
| 7.5 | mg MB | 99.96% | 2.69E+05 | 3.01E+05 |
| (0.06%) | | | | |
| 1 | mg MB | 96.76% | 3.20E+06 | 1.46E+06 |

(0.008%)

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An antimicrobial bone cement composition comprising
a) a plurality of metallic nanoparticles;
b) one or more photosensitizers;
c) bone cement;
wherein the metallic nanoparticles, one or more photosensitizers, and the radical initiator are incorporated into the bone cement to form an antimicrobial bone cement composition;
wherein the antimicrobial bone cement composition exhibits antimicrobial activity under light and dark conditions.

2. The antimicrobial bone cement composition of claim 1, wherein the plurality of metallic nanoparticles are selected from a group consisting of zinc oxide, zinc, gold, silver, copper, cupric oxide, magnesium oxide, iron (II,III) oxide, graphene oxide, titanium dioxide, tungsten (VI) oxide nanoparticles, alloys of two or more of these metals, or a combination thereof.

3. The antimicrobial bone cement composition of claim 1, wherein neither the plurality of metallic nanoparticles nor the one or more photosensitizers leaches from the composition.

4. The antimicrobial polymer composition of claim 1, wherein the one or more photosensitizers comprises at least 0.001% by weight of the total polymer.

5. The antimicrobial polymer composition of claim 1, wherein the metallic nanoparticles comprise about 0.1% to about 10% by weight of the total polymer.

6. The antimicrobial bone cement composition of claim 1, wherein the composition generates reactive oxygen species in the absence of an external light source.

7. The antimicrobial bone cement composition of claim 1, wherein the one or more photosensitizers are selected from the group consisting of toluidine blue O, methylene blue, dimethylmethylene blue, zinc phthalocyanine, silicon phthalocyanine, aluminum phthalocyanine, rose bengal, porphyrin, haematoporphyrin, deuteroporphyrin, tin chlorin e6, poly-lysine derivatives of tin chlorin e6, m-tetrahydroxyphenyl chlorin, benzoporphyrin derivatives, tin etiopurpurin, bacteriochlorins, phenazines, acriflavine, proflavin, acridine orange, aminacrine, texaphyrins, cyanines including merocyanine 540, anthracyclins including adriamycin epirubicin, pheophorbides, sapphyrins, fullerene, perylenequinonoid pigments including hypericin and hypocrellin, gilvocarcins, terthiophenes, benzophenanthridines, tris(2,2′-bipyridine)ruthenium(II) hexafluorophosphate, quercetin hydrate, curcumin, Zn(II) phthalocyanine, eosin-Y disodium salt, psoralens and riboflavin arianor steel blue, tryptan blue, crystal violet, azure blue cert, azure B chloride, azure 2, azure A chloride, azure B tetrafluoroborate, thionin, azure A eosinate, azure B eosinate, azure mix sicc, and azure II eosinate, or a combination thereof.

8. The antimicrobial bone cement composition of claim 1, wherein the photosensitizer is tris(2,2′-bipyridine)ruthenium(II) hexafluorophosphate, quercetin hydrate, curcumin, Zn(II) phthalocyanine, crystal voilet, azure-A, eosin-Y disodium salt, methylene blue, rose bengal, or a combination thereof.

9. The antimicrobial bone cement composition of claim 1, comprises zinc oxide and methylene blue, wherein the zinc oxide and methylene blue are dispersed within the bone cement.

10. The antimicrobial bone cement composition of claim 1, wherein the plurality of nanoparticles comprises nanoparticles of less than 100 nm in diameter.

11. The antimicrobial bone cement composition of claim 1, further comprising a radiopaque filler to make the implantable device easily visible under X-rays.

12. A method for making an antimicrobial bone cement composition of claim 1 comprising the steps of:
a) providing a cement powder comprising:
i) a polymer powder;
ii) a polymerization initiator;
iii) a plurality of metallic nanoparticles; and
iv) one or more photosensitizers;
b) providing a liquid component comprising a polymerizable monomer and an activator;
c) combining the cement powder with the liquid component to form a bone cement mixture;
d) transferring the bonce cement mixture into a mold; and
e) allowing the bone cement mixture to cure;
wherein the cured bone cement mixture exhibits antimicrobial activity under both light and dark conditions.

13. The method of claim 12, wherein the initiator is a peroxide.

14. The method of claim 12, wherein the cement powder further comprises a radiopaque filler to make the implantable device easily visible under X-rays.

15. The method of claim 12, wherein transferring the antimicrobial bone cement mixture into a mold is completed within 10 minutes.

16. The method of claim 12, wherein the mold is a bone cavity within a subject in need of a bone cement composition.

17. The method of claim 12, wherein the antimicrobial bone cement is transferred into a mold via injection.

18. The method of claim 12, wherein the antimicrobial bone cement is used in orthopedic or dental applications.

19. A kit for producing the antimicrobial bone cement composition of claim 1, comprising: polymethyl methacrylate, an initiator, a plurality of metallic nanoparticles, one or more photosensitizers, methyl methacrylate, and dimethyl-p-toluidine.

20. The kit of claim 19, further comprising one or more additives selected from zirconium oxide, barium sulfate, vitamin E, chemotherapy agents, or a combination thereof.
